(19) United States
(12) Patent Application Publication
(10) Pub. No.: US 2009/0298879 A1
(43) Pub. Date: Dec. 3, 2009

(54) Impurities of Donepezil

(75) Inventors: Irena Krizmanic, Zagreb (HR); Lidija Lerman, Zagreb (HR); Zrinka Samardzic, Zagreb (HR); Katarzyna Kaczorowska, Zagreb (HR); Andrzej Manikowski, Zagreb (HR); Barbara Zioro, Zagreb (HR); Ivica Grebenar, Zagreb (HR); Jasna Dogan Koruznjak, Zagreb (HR)

Correspondence Address: MERCHANT & GOULD PC, P.O. BOX 2903, MINNEAPOLIS, MN, 55402-0903, US

(73) Assignee: PLIVA Hrvatska d.o.o., Zagreb (HR)

(21) Appl. No.: 12/301,396

(22) PCT Filed: May 18, 2007
(86) PCT No.: PCT/GB2007/001872

§ 371 Date: May 6, 2009

(30) Foreign Application Priority Data
May 18, 2006 (GB) 0609835.4

(51) Int. Cl. A61K 31/445; C07C 211/01; C07D 211/06; A61P 25/00; G01N 30/02

(52) U.S. Cl. 514/319; 564/384; 546/206; 73/61.55

(57) ABSTRACT

The present invention relates to impurities of donepezil, or a pharmaceutically acceptable salt thereof, which may be produced during synthesis and storage of donepezil, or a pharmaceutically-acceptable salt thereof, and to methods of identifying such impurities. The invention also relates to the use of these impurities in analytical techniques and to product that contain certain low levels of these impurities in particular the following impurities are disclosed: formula (I) and formula (II).

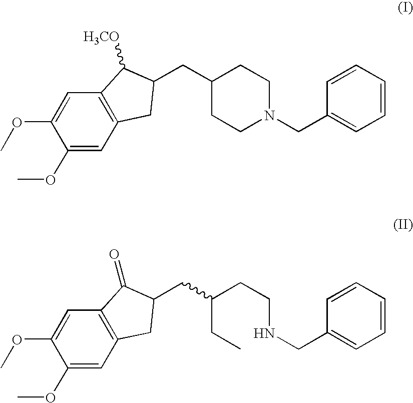

### FIELD OF INVENTION

The present invention relates to impurities of donepezil, or a pharmaceutically-acceptable salt thereof, which may be produced during synthesis and storage of donepezil, or a pharmaceutically-acceptable salt thereof, and to methods of identifying such impurities. The invention also relates to the use of these impurities in analytical techniques and to products that contain certain low levels of these impurities.

### BACKGROUND

Donepezil (1-benzyl-4-(5,6-dimethoxyindanone-2-yl)methylpiperidine) is a pharmaceutically active compound. Its structure is shown in Formula A

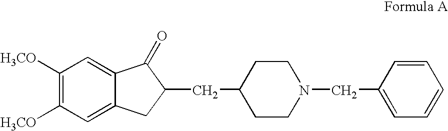

Donepezil belongs to a group of selective acetylcholinesterase inhibitors used for the treatment of mild to moderate Alzheimer's disease. Symptoms of the disease include memory loss, confusion, personality changes, disorientation and loss of language skills.

Several methods for the preparation of intermediates useful in the synthesis of donepezil have been described.

It is well known in the art that, for human administration, safety considerations require the establishment, by national and international regulatory authorities, of very low limits for identified, but toxicologically uncharacterised impurities, before an active pharmaceutical ingredient (API) product is commercialised. Typically, these limits are less than about 0.15 percent by weight of each impurity. Limits for unidentified and/or uncharacterised impurities are obviously lower, typically less than 0.1 percent by weight. Specific standards can be applied to certain drugs where a pharmacopoeia monograph has been established for that drug. Typically, for impurities that are present in an amount of greater than 0.1 percent by weight, the impurity should be fully identified and characterised.

Therefore, in the manufacture of active pharmaceutical ingredients (APIs) knowledge of the purity of the API, such as donepezil, is required before commercialisation, as is the purity of the API in the manufactured formulated pharmaceutical product.

Impurities introduced during commercial manufacturing processes must be limited to very small amounts and are preferably substantially absent. For example, the ICH Q7A guidance for API manufacturers requires that process impurities be maintained below set limits by specifying the quality of raw materials, controlling process parameters, such as temperature, pressure, time, and stoichiometric ratios, and including purification steps, such as crystallisation, distillation, and liquid-liquid extraction, in the manufacturing process.

The direct product of a chemical reaction is rarely a single compound with sufficient purity to comply with pharmaceutical standards. Intermediates and by-products will, in most cases, be present with the API. At certain stages during processing of an API, such as donepezil, it must be analysed for purity, typically, by HPLC or TLC analysis, to determine the presence of any intermediates or by-products. The API need not be absolutely pure, as absolute purity is a theoretical ideal that is typically unattainable. Rather, purity standards are set with the intention of ensuring that an API is as free of impurities as possible, and, thus, is as safe as possible for human use. As discussed above, in the United States, the Food and Drug Administration guidelines recommend that the amounts of some impurities be limited to less than 0.1% wt.

Generally, by-products and intermediates (collectively hereinafter defined as “impurities”) are identified spectroscopically and/or with another physical method, and then associated with a peak position, such as that in a chromatogram, or a spot on a TLC plate. [Strobel p. 953, Strobel, H. A.; Heineman, W. R., Chemical Instrumentation: A Systematic Approach, 3rd ed. (Wiley & Sons: New York 1989)]. Thereafter, the impurity can be identified, e.g., by its position in the chromatogram, where the position in a chromatogram is conventionally measured in minutes between injection of the sample on the column and elusion of the particular component through the detector.

The relative position in the chromatogram is known as the “retention time.” The retention time can vary about a mean value based upon the condition of the instrumentation, as well as many other factors. To mitigate the effects such variations have upon accurate identification of an impurity, practitioners use the “relative retention time” (“RRT”) to identify impurities. (Strobel p. 922). The RRT of an impurity is its retention time divided by the retention time of “a reference marker”. The reference marker can be the API itself, or it may be a compound other than the API that is added to, or present already in the mixture, in an amount sufficiently large to be detectable and sufficiently low so as not to saturate the column, and to use that compound as the reference marker for determination of the RRT.

Those skilled in the art of drug manufacturing research and development understand that a compound in a relatively pure state can be used as a “reference standard”. A reference standard is similar to a “reference marker”, which is used for qualitative analysis only, but is used to quantify the amount of the compound of the reference standard in an unknown mixture, as well. A reference standard is an “external standard,” when a solution of a known concentration of the reference standard and an unknown mixture are analysed using the same technique. [Strobel p. 924, Snyder p. 549, Snyder, L. R.; Kirkland, J. J. Introduction to Modern Liquid Chromatography, 2nd ed. (John Wiley &Sons: New York 1979)]. The amount of the compound in the mixture can be determined by comparing the magnitude of the detector response. See also U.S. Pat. No. 6,333,198, where the passages relating to the techniques for determining response factors are incorporated herein by reference.

The reference standard can also be used to quantify the amount of another compound in the mixture if a “response factor”, which compensates for differences in the sensitivity of the detector to the two compounds, has been predetermined (Strobel p. 894). For this purpose, the reference standard is added directly to the mixture, and is known as an “internal standard” (Strobel p. 925, Snyder p. 552).

The reference standard can serve as an internal standard when, without the deliberate addition of the reference standard, an unknown mixture contains a detectable amount of the reference standard compound using the technique known as “standard addition”. In the “standard addition technique”, at least two samples are prepared by adding known and differing amounts of the internal standard (Strobel pp. 391-393, Snyder pp. 571, 572). The proportion of the detector response due to the reference standard present in the mixture without the addition can be determined by plotting the detector response against the amount of the reference standard added to each of the samples, and extrapolating the plot to zero concentration of the reference standard (See, e.g., Strobel, FIG. 11.4, p. 392).

WO06030249 mentions MS and HPLC as methods which can be used for identifying impurities and determining their concentration, respectively, during stability examinations of tablets containing donepezil hydrochloride. However it does not provide a detailed description of said methods and ways of conducting an identification of impurities and determining their concentration using said methods.

WO06015338 describes a process for preparing donepezil hydrochloride form I whilst stating maximum area-percentage values of organic compound impurities present in product. HPLC is mentioned as a method used for determining said values.

Reddy et al., (“Identification and characterization of potential impurities of donepezil”, Journal of Pharmaceutical and Biomedical Analysis, 35, 2004, 1047-1058) discloses one possible way of conducting an HPLC analysis for identification of donepezil impurities. The method described by Reddy et al. employs different equipment and reagents from the method disclosed herein.

### BRIEF DESCRIPTION OF THE DRAWING

FIG. 1 shows a HPLC chromatogram of a donepezil sample comprising impurities described herein.

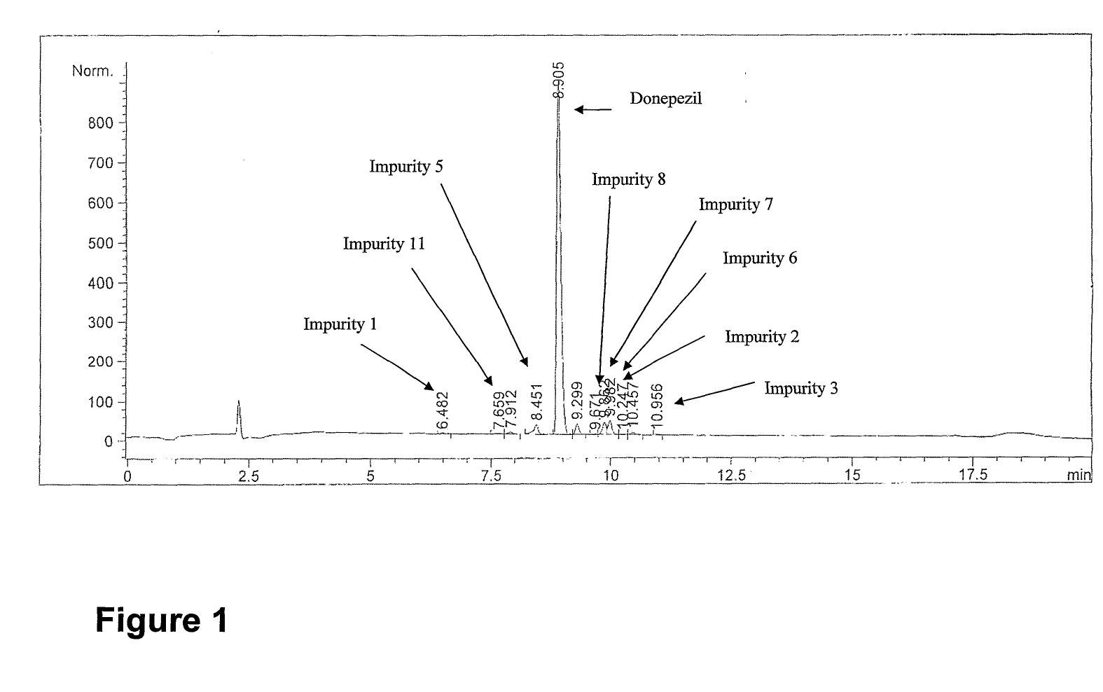

### DESCRIPTION OF THE INVENTION

As is known by those skilled in the art, the management of process impurities is greatly enhanced by understanding their chemical structures, and by identifying the parameters that influence the amount of impurities in the final product. Moreover, it is important that the presence or absence of an impurity in a product be identified in any quality control process in order to ensure that the process complies with the required standards set down in the regulatory approval of that product prior to it being released for commercial sale.

Like any synthetic compound, donepezil, or a pharmaceutically-acceptable salt thereof, can contain impurities that can come from many sources. They can be degradation products of donepezil, unreacted intermediate(s) used in the preparation of donepezil or by-products of the reactions used to prepare donepezil.

The invention relates generally to the identification of impurities found in donepezil, or a pharmaceutically-acceptable salt thereof, and to the use of these impurities as reference markers in analytical methods for measuring the quality of the donepezil product. The invention also relates to some of these impurities as novel chemical entities. The invention also relates to the use of the isolated impurity as a reference marker. The invention also relates to mixtures of donepezil, or a pharmaceutically-acceptable salt thereof, containing such impurities below certain levels.

We have identified a number of impurities that are found in the manufacture of donepezil, or a pharmaceutically-acceptable salt thereof. A certain number of these impurities are fully characterised below, in Tables 1, 2, and 3. The compounds of Table 2, compounds 5, 6 and 7 have not previously been characterised or identified. Any one or more of the impurities identified in Table 1 or Table 2 or Table 3 may be used as a reference marker. In some embodiments, the reference marker is donepezil. Use of a compound as a reference marker requires recourse to a sample of that compound which is substantially pure. As used herein, the term “substantially pure” when used in connection with a compound refers to the compound as being at least 90% wt pure. In some embodiments a substantially pure compound is at least at least 91, 92, 93, 94, 95, 96, 97, 98 or 99% wt pure.

When an impurity of donepezil or another compound is used as a reference marker, it may be used in an isolated form. An isolated compound, such as an isolated impurity, refers to a compound which has been isolated from a mixture by removal of some, most or all of the other components of the mixture. In some embodiments, the isolated compound may be substantially pure.

A “reference marker” is used in qualitative analysis to identify components of a mixture based upon their position, e.g., in a chromatogram such as on a Thin Layer Chromatography (TLC) plate (Strobel pp; 921, 922, 953). For this purpose, the compound does not necessarily have to be added to the mixture if it is present in the mixture. If the amount of reference marker is quantified, it can be used in a quantitative analysis as a reference standard. As used herein, the term “reference standard” refers to a compound that may be used both for quantitative and qualitative analysis of an active pharmaceutical ingredient. In this disclosure the impurities identified in Table 1 or Table 2 or Table 3 may be used as a reference standard.

Therefore, in one aspect, the invention includes the use of any one of the compounds disclosed in Tables 1, 2 and 3 as a reference marker in methods for determining the identity of impurities in a sample of donepezil, or a pharmaceutically-acceptable salt thereof, or as a reference standard in methods of determining the identity and amount of impurities in a sample of donepezil, or a pharmaceutically-acceptable salt thereof.

For example, the HPLC retention time of the compound allows a relative retention time to be determined, thus making qualitative analysis possible. The concentration of the compound in solution before injection into an HPLC column allows the areas under the HPLC peaks to be compared, thus making quantitative analysis possible.

Reference standards are described in general terms above. However, as will be understood by those skilled in the art, a detector response can be, for example, the peak heights or integrated peak areas of a chromatogram obtained, e.g., by UV or refractive index detection, from the eluent of an HPLC system or, e.g., flame ionisation detection (FID) or thermal conductivity detection, from the eluent of a gas chromatograph, or other detector response, e.g., the UV absorbance of spots on a fluorescent TLC plate. The position of the reference standard may be used to calculate the relative retention time for donepezil and impurities of donepezil.

TABLE 1
Impurities known in literature

| | | | Retention | |
|---|---|---|---|---|
| | | | Time | |
| | | Mass | relative | |
| Impurity | | Spectroscopy | to | Type of |
| Number | Structure | (m/z) | donepezil | impurity |
| 1 | 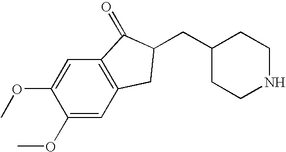 | 289 | 0.74 ± 0.01 | By- product |
| 2 | 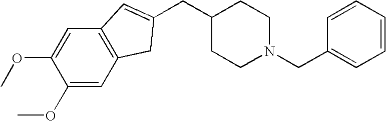 | 363 | 1.16 ± 0.01 | By- product |
| 3 | 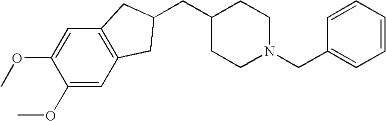 | 365 | 1.20 ± 0.02 | By- product |
| 4⁽¹⁾ | 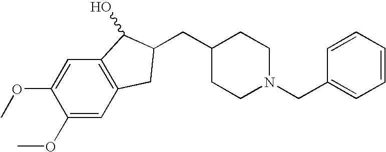 | 381 | 0.94 ± 0.01 | By- product |

⁽¹⁾Wiggly line in structure represents the presence of two diastereoisomers of the impurity. The RRTs of both of them are 0.94 ± 0.01

TABLE 2
Impurities not known in literature

| | | | Retention | |
|---|---|---|---|---|
| | | | Time | |
| | | Mass | relative | |
| Impurity | | Spectroscopy | to | Type of |
| Number | Structure | (m/z) | donepezil | impurity |
| 5⁽¹⁾ | 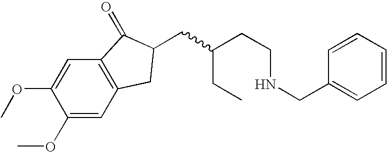 | 381 | from 1.10 ± 0.01 to 1.12 ± 0.01 | |
| 6 | Unknown | 385 | 1.08 ± 0.01 | |
| 7⁽²⁾ | 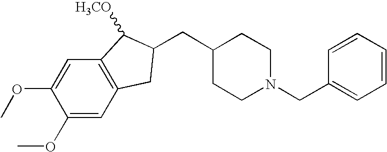 | 395 | 1.07 ± 0.02 | By- product |

⁽¹⁾Wiggly line in structure represents the presence of two diastereoisomers of the impurity. RRT of one of them is 1.10 ± 0.01 and of the other one is 1.12 ± 0.01.
⁽²⁾Wiggly line in structure represents the presence of two diastereoisomers of the impurity. The RRTs of both of them are 1.07 ± 0.02.

TABLE 3
Intermediates

| | | | Retention | |
|---|---|---|---|---|
| | | | Time | |
| | | Mass | relative | |
| Impurity | | Spectroscopy | to | Type of |
| Number | Structure | (m/z) | donepezil | impurity |
| 8⁽³⁾ | 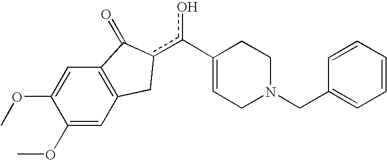 | 391 | 1.42 ± 0.01 | Intermediate |
| 9 | 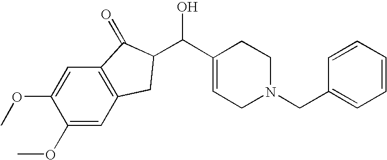 | 393 | 0.89 ± 0.01 | Intermediate |
| 10 | 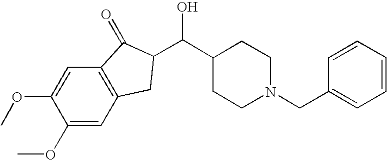 | 395 | 0.86 ± 0.01 | Intermediate |

⁽³⁾The dashed line in the structure indicates the presence of either or both of the ketone/enol tautomers.

Retention times relative to donepezil given in Tables 1, 2 and 3 and below in the text are measured by chromatographic analysis using commercially available, HPLC and TLC equipment such as described in Example 1 and appropriate UV and MS detectors. Preferred analysis is HPLC.

In another aspect, there are provided methods of identifying an impurity in a sample of donepezil, or a pharmaceutically-acceptable salt thereof, the method comprising performing steps (a) and (b) in either order;

(a) carrying out a chromatographic analysis on the reference sample, comprising one of the above impurities (“reference marker”) and donepezil, or a pharmaceutically-acceptable salt thereof, to determine the relative retention time of the reference marker compared to donepezil, or a pharmaceutically-acceptable salt thereof;
(b) carrying out the same chromatographic analysis as in step (b) on the sample of donepezil, or a pharmaceutically-acceptable salt thereof, to determine the relative retention time of any impurities present in the sample compared to donepezil, or a pharmaceutically-acceptable salt thereof;
and comparing the relative retention times determined in steps (a) and (b); where, if the relative retention times determined in steps (a) and (b) are substantially the same, the impurity of donepezil, or a pharmaceutically-acceptable salt thereof, if the sample is identified as being the same as the reference marker.

In some embodiments of the methods, the impurity is selected from one or more of compounds 5, 6 and 7. In other embodiments, the methods further comprise determining the relative retention time of one or more impurities of donepezil selected from compounds 1, 2, 3, 4, 8, 9, and 10.

In another aspect, the invention provides a method of determining the amount of an impurity in a sample of donepezil, or a pharmaceutically-acceptable salt thereof, the method comprising performing steps (a) and (b) in either order:

(a) subjecting a reference sample to a HPLC analysis wherein the reference sample comprises a known amount of at least one of the impurities (“reference standard”) and donepezil, or a pharmaceutically-acceptable salt thereof, determining the relative retention time of the reference standard compared to donepezil, or a pharmaceutically-acceptable salt thereof; and measuring the area of the HPLC peak associated with the reference standard;
(b) carrying out the same HPLC analysis as in step a) on the sample of donepezil, or a pharmaceutically-acceptable salt thereof, and identifying each HPLC peak with each and every impurity by reference to the relative retention times determined in step (a)
and calculating the amount of each impurity in the donepezil, or a pharmaceutically-acceptable salt thereof, by reference to the area of the HPLC peak in the sample against the area of the HPLC peak associated with the same impurity in step (b).

In another embodiment of methods of determining the amount of an impurity in a sample of donepezil, or a pharmaceutically-acceptable salt thereof, the method comprising performing steps (a) and (b) in either order:

(a) subjecting a sample of donepezil, or a pharmaceutically-acceptable salt thereof, comprising an unknown concentration of at least one impurity of donepezil and a sample of a known concentration of, at least one, impurity of donepezil, as described above, to a chromatographic analysis;
(b) measuring the area of the at least one impurity of donepezil peaks obtained from the sample of donepezil, or a pharmaceutically-acceptable salt thereof and from the sample of the at least one impurity of donepezil;
and calculating the concentration of the at least one impurity of donepezil in the sample of donepezil, or a pharmaceutically-acceptable salt thereof, from the measured area.

In still another embodiment of methods of determining the amount of an impurity in a sample of donepezil, or a pharmaceutically-acceptable salt thereof, the method comprising performing steps (a) and (b) in either order:

(a) subjecting an aliquot of a reference solution of a known concentration of substantially pure donepezil, or a pharmaceutically-acceptable salt thereof, and an aliquot of reference solution of a known concentration of one substantially pure isolated impurity of donepezil to chromatographic analysis and determining the response factors of donepezil, or a pharmaceutically-acceptable salt thereof, and the impurity of donepezil;
(b) subjecting a solution of known overall concentration of the donepezil, or a pharmaceutically-acceptable salt thereof, to chromatographic analysis under substantially the same conditions used in step (a);
and calculating the amount of impurity of donepezil in the solution using the respective peak areas and the response factors.

It will be understood that steps (a) and (b) may be carried out in either order or at the same time.

In another aspect, the invention provides processes for preparing a pharmaceutical composition comprising donepezil, or a pharmaceutically-acceptable salt thereof, which comprise formulating donepezil or a pharmaceutically acceptable salt thereof, tested according to above presented methods for identifying impurities and for determining the amount of impurities in a sample of donepezil or a pharmaceutically-acceptable salt thereof.

In the methods described herein, the sample of donepezil, or a pharmaceutically-acceptable salt thereof, can be bulk donepezil or a pharmaceutically-acceptable salt thereof, or can be donepezil or a pharmaceutically-acceptable salt thereof, separated from one or more pharmaceutical dosage forms comprising donepezil. In some embodiments, the sample of donepezil or a pharmaceutically-acceptable salt thereof, is taken from a batch or production lot of donepezil. Preferably, the amount of each impurity of the sample of donepezil is determined to be less than 0.15 area-% as judged by HPLC, and the batch can be released for sale.

In one aspect of the invention, there are provided one or more isolated impurities of donepezil, or a pharmaceutically-acceptable salt thereof, such as those given in Table 2. For example, an isolated impurity of donepezil, or a pharmaceutically-acceptable salt, can have a retention time, relative to donepezil, in between 1.10±0.01 and 1.12±0.01, as measured by a method of identifying an impurity in a sample of donepezil described above and in Example 1, depending on a present diastereomer thereof. An isolated impurity of donepezil, or a pharmaceutically-acceptable salt can also have the following structure

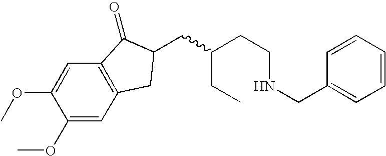

and a diastereomer thereof

Another isolated impurity of donepezil, or a pharmaceutically-acceptable salt thereof can have a retention time, relative to donepezil, of 1.08±0.01 as measured by a method of identifying an impurity in a sample of donepezil described above and in Example 1.

Still another isolated impurity of donepezil, or a pharmaceutically-acceptable salt thereof, can have a retention time of both diastereomers, relative to donepezil, of 1.07±0.02 as measured by a method of identifying an impurity in a sample of donepezil described above and in Example 1. The isolated impurity of donepezil, or a pharmaceutically-acceptable salt thereof can have the following structure

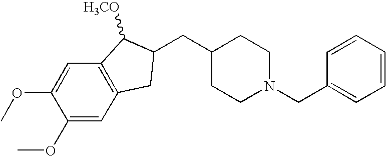

and a diastereomer thereof.

In some embodiments, one or more isolated impurities of donepezil, or a pharmaceutically-acceptable salt thereof, described above, comprise 0.03 area-% to 2 area-% donepezil, or a pharmaceutically-acceptable salt thereof, as judged by HPLC, preferably 0.05 area-% to 1.5 area-% donepezil, and preferably 0.06 area-% to 1.0 area-% donepezil. In other embodiments, the isolated impurity of donepezil or a salt thereof is as defined in Table 2, and is at least 90 area-% pure as judged by HPLC, or at least 91, 92, 93, 94, 95, 96, 97, 98, or 99 area-% pure as judged by HPLC.

In another aspect, the invention provides donepezil, or pharmaceutically-acceptable salt thereof, containing one or more impurities from Table 1 or Table 2 or Table 3 as characterised above, wherein each impurity is present in an amount not exceeding more than 0.5 percent by weight (% wt), and preferably not more than 0.4, 0.3, 0.2, 0.1, 0.05, 0.04, 0.03, 0.02 or 0.01% wt, of the total % wt of donepezil, whether in the form of donepezil or its pharmaceutically-acceptable salt.

Pharmaceutical compositions or formulations of donepezil may be prepared which comprise donepezil or a pharmaceutically-acceptable salt thereof, at least one pharmaceutically-acceptable excipient, and at least one or more impurities from Tables 1 to 3, wherein each impurity is present in an amount not exceeding more than 0.5% wt (preferably not more than 0.4, 0.3, 0.2, 0.1, 0.05, 0.04, 0.03, 0.02 and 0.01% wt) of the total % wt of donepezil present in the formulation, whether in the form of donepezil or its pharmaceutically-acceptable salt. In some embodiments, at least one impurity is selected from Table 2 and, optionally, at least one or more impurities from Tables 1 and 3.

Pharmaceutical compositions described herein may be prepared by various methods. The methods include mixing donepezil or a pharmaceutically acceptable salt thereof as described herein (i.e., donepezil having one or more impurities as described herein), with at least one pharmaceutically acceptable excipient or carrier and optionally forming the mixture into a pharmaceutical dosage form (for example a tablet). In another embodiment, there are provided processes for preparing a pharmaceutical composition including donepezil or a pharmaceutically acceptable salt thereof which comprise combining donepezil or a pharmaceutically acceptable salt thereof, tested according to any of the methods described herein, with an excipient and/or carrier.

A pharmaceutical formulation comprising donepezil, or pharmaceutically-acceptable salt thereof, at least one pharmaceutically-acceptable excipient therefore, and at least one or more impurities from 1 to 11, as described above, wherein each impurity is present in an amount not exceeding more than 0.5% wt (preferably not more than 0.4, 0.3, 0.2, 0.1, 0.05, 0.04, 0.03, 0.02 and 0.01% wt) of the total % wt of donepezil, or a pharmaceutically-acceptable salt thereof, present in the formulation, whether in the form of donepezil or its pharmaceutically-acceptable salt.

The release criteria for commercial sale of API can be established with reference to a particular amount or concentration of a reference standard in the bulk product. Detection and quantification of the reference standard in the API of a pharmaceutical dosage form can serve as a measure of the shelf-life of the pharmaceutical dosage form. That is, detection of the reference standard at some concentration signals that the API has begun to deteriorate and that efficacy of the API may be compromised. As used herein, HPLC refers to the well-known technique of high-performance liquid chromatography, also referred to as high pressure liquid chromatography. HPLC can be applied to detection and quantification of components of a mixture, for example detection and quantification of impurities in a principal compound such as an active pharmaceutical ingredient (API).

Detection and especially quantification of components of a mixture can be accomplished with the use of response factors. The response of a detector in HPLC (e.g. UV detectors or refractive index detectors) can be and typically is different for each compound eluting from the HPLC column. Response factors are known to account for this difference in the response signal of the detector to different compounds eluting from the column.

The response factor is calculated as follows. If Y is the primary or principal component, the relative response factor of compound X, i.e., an impurity in Y and especially a reference marker for the purity of Y, can be expressed as:

Ry/x=[Mx/My]/[Ax/Ay]

where Mx and My are the known molar amounts or concentrations of X and Y in a standard solution (a solution having known amount of X and Y) and Ax and Ay are the detector responses, for example peak areas in HPLC, for species X and Y respectively.

Therefore, in a solution having a known total amount of sample but unknown relative amounts of Y and X:

M′x/M′y=Ry/x*[A′x/A′y]

where M′x and M′y are the amounts of X and Y respectively in the solution, A′x and A′y are the associated detector responses for X and Y. Determination of response factors requires access to samples of substantially pure X and Y especially when X is a reference marker for the purity of Y.

Determination of the purity of donepezil, or a pharmaceutically-acceptable salt thereof, in a pharmaceutical dosage form that includes donepezil, or a pharmaceutically-acceptable salt thereof, for example to evaluate shelf life and residual potency, requires that the donepezil, or a pharmaceutically-acceptable salt thereof, be separated from excipients and other intentionally added ingredients in the dosage form. This can be accomplished by, for example, combining a suitable quantity of the dosage form, comminuted if desired, with a suitable solvent. Suitable solvents dissolve donepezil and impurities therein, but do not dissolve excipients and other intentionally added ingredients and can be selected from water, methanol and acetonitrile.

Detection and especially quantification of components of a mixture by HPLC requires that the peaks for the components be sufficiently separated (resolved). This can be checked by performing a system suitability check to determine the resolution factors according to a method such as described below.

The well-known technique of thin layer chromatography (TLC) can also be applied to assessment of the purity of an API. In this case, presence of an impurity, e.g. a reference marker, is established by the presence of a suitably-visualised “spot” at the same, simultaneously determined relative position, RF, (relative to the solvent front) of the reference marker.

### EXAMPLES

### Example 1

The impurities were identified using a combined HPLC and MS analysis according to method A.

The method A:

Apparatus and Working Conditions:

An analytical HPLC instrument with UV and MS detector

Column: 150×4.6 mm ID, S-3 μm, 12 nm (such as provided by YMC-Pack Pro C18

Column temperature: 35° C.

Flow rate: 1 ml/min

Detector:
a) DAD: wavelength: 240 nm, Bw 8 nm; Ref. Signal 360 nm, Bw 100 nm
b) MSD/Ion Trap: ES ionization; Capillary 3500 V, Nebuliser 40.0 psi, Dry Gas 10.0 l/min, Dry Temp. 350° C., Skimmer 40 V, Cap. Exit 166 V, Target 30000, Max. Accu. Time 300.00 ms

Injection volume: 3 μl

Mobile phase: A: B=90% v/v: 10% V/V
A) 40 mM NH₄OAc (pH=5.0)
B) CH₃CN

Elution: Gradient program: 0 min→10% B, 15 min→90% B, 15.10 min→90% B,
20 min→10% B

Stop time: 20 min

Reagents:

Acetonitrile; gradient grade for chromatography

Ammonium acetate; gradient grade for chromatography

Water; purified

Preparation of Sample Solution:

Weigh accurately about 1.0 mg of donepezil standard and dissolve it in 1 ml of dilution solution (mixture of acetonitrile and water in the ratio of 95:5).

Using the HPLC-MS methodology described above, the impurities of donepezil were determined based upon HPLC relative retention times, being relative to donepezil.

### Example 2

### Preparation of 2-[(1-benzyl-1,2,3,6-tetrahydro-pyridin-4-yl)-hydroxy-methyl]-5,6-dimethoxy-indan-1-one (Impurity 10)

To an orange suspension of 1-benzyl-4-(5,6-dimethoxy-1-oxo-indane-2-carbonyl)-pyridinium bromide (20 g, 0.043 mol) in 510 cm³ of methanol, cooled down to about 10° C., a solution of sodium borohydride (4.04 g, 0.107 mol) and KOH (0.8 g, 0.014 mol) in 90 cm³ of water was continuously dripped over 30 minutes. The internal temperature was kept in the range of 10-18° C. The reaction mixture was stirred for the next 10 min and 200 ml of water was added followed by acidification with 8% hydrochloric acid to bring pH to ˜4, keeping the temperature below 20° C. Methanol was removed under reduced pressure at 50° C., and the resulting aqueous liquor was cooled down to 20° C. and treated with 60 cm³ of dichloromethane and 10% KOH until the pH was brought to ˜8, keeping temperature below 20° C. The products were extracted with all together 240 cm³ of dichloromethane (4×60 cm³). The combined organic layers were evaporated under reduced pressure to provide an oily residue, which was dissolved in 40 cm³ of hot iso-propanol. The solution was cooled down to room temperature and next was kept for 5 hours at 8° C. The white crystals were filtered off, washed with cooled iso-propanol and dried to obtain 11 g of product.

### Example 3

### Preparation of Donepezil Hydrochloride

1.2 g of 2-[(1-benzyl-1,2,3,6-tetrahydro-pyridin-4-yl)-hydroxy-methyl]-5,6-dimethoxy-indan-1-one (from Example 2) was dissolved in 8 ml iso-propanol and next acidified with 0.85 ml ˜24% HCl/EtOH diluted in 3.5 ml iso-propanol. The mixture was stirred under reflux for 1 h and was allowed to cool down to room temperature. The obtained thick slurry was next filtered off and the product was washed with 2×3 ml of iso-propanol and 5 ml of acetone and dried. 1 g of obtained compound and 80 mg of Pt/C catalyst were suspended in methanol and hydrogenated under the pressure of 10 bar and at temperature of 35° C. until the hydrogenation reaction was substantially complete. The progress of the reaction was monitored by the HPLC-MS-UV method A. After the catalyst was filtered off, the filtrate was concentrated under reduced pressure. The concentrated solution was cooled down and ethyl acetate was added drop wise. The resulting suspension was filtered, washed and dried to produce 0.6 g of crude donepezil hydrochloride.

### Example 4

### Recrystallisation of Donepezil Hydrochloride

1.7 g of crude donepezil hydrochloride was dissolved in mixture of 8.6 ml of methanol and 0.5 ml of deionized water at room temperature. The resulting solution was cooled down to 0-5° C. Then 48 ml of tert-butyl methyl ether was added drop wise during 35 minutes. Stirring of the resulting suspension was continued for 30 minutes at 0-5° C. The precipitate was filtered, washed with tert-butyl methyl ether and dried to yield 1.21 g of donepezil hydrochloride.

1. A method of identifying an impurity in a sample of donepezil, or a pharmaceutically-acceptable salt thereof, selected from one or more of the following compounds: 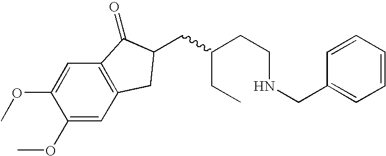
or a diastereomer thereof, and 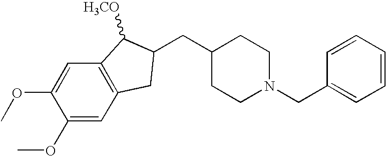
or a diastereomer thereof,
the method comprising performing steps (a) and (b) in either order
(a) carrying out a chromatographic analysis on a reference sample, comprising one of the above impurities (“reference marker”) and donepezil, or a pharmaceutically-acceptable salt thereof, to determine a relative retention time of the reference marker compared to donepezil, or a pharmaceutically-acceptable salt thereof;
(b) carrying out the same chromatographic analysis as in step a) on the sample of donepezil, or a pharmaceutically-acceptable salt thereof, to determine a relative retention time of any impurities present in the sample compared to donepezil, or a pharmaceutically-acceptable salt thereof;
and comparing the relative retention times determined in steps (a) and (b); where, if the relative retention times determined in steps (a) and (b) are substantially the same, the impurity of donepezil, or a pharmaceutically-acceptable salt thereof, in the sample is identified as being the same as the reference marker.

2. The method according to claim 1 wherein the chromatographic analysis is HPLC or TLC analysis.

3. The method according to claim 2, wherein in both steps (a) and (b) HPLC is carried out.

4. A method claims according to claim 1 wherein the relative retention time of one or more of following impurities of donepezil is also determined: 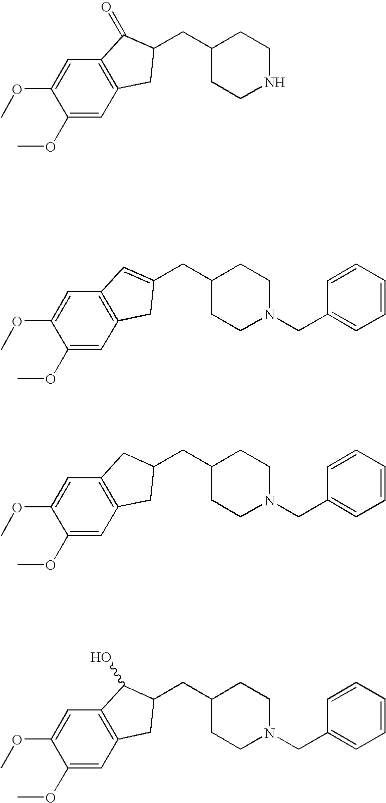
or a diastereomer thereof,

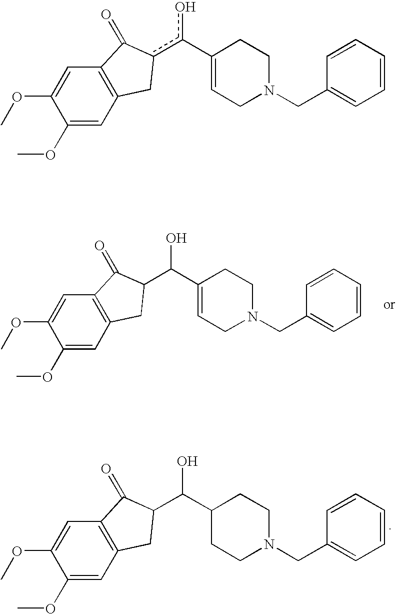

5. A method of determining an amount of an impurity in a sample of donepezil, or a pharmaceutically-acceptable salt thereof, selected from one or more of the following compounds: 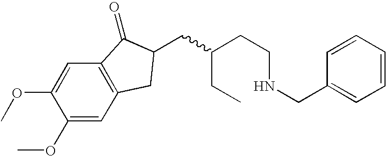
or a diastereomer thereof, and 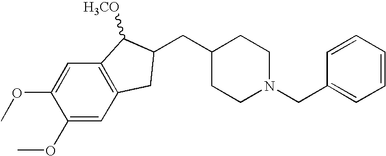
or a diastereomer thereof
the method comprising performing steps (a) and (b) in either order:
(a) subjecting a reference sample to a HPLC analysis wherein the reference sample comprises a known amount of at least one impurity (“reference standard”) and donepezil, or a pharmaceutically-acceptable salt thereof, determining a relative retention time of the reference standard compared to donepezil, or a pharmaceutically-acceptable salt thereof, and measuring an area of an HPLC peak associated with the reference standard;
(b) carrying out the same HPLC analysis as in step (a) on the sample of donepezil, or a pharmaceutically-acceptable salt thereof, and identifying and measuring an area of each HPLC peak associated with each and every impurity by reference to the relative retention times determined in step (a);
and calculating an amount of each impurity in the donepezil, or a pharmaceutically-acceptable salt thereof, by reference to the area of the HPLC peak in the sample against the area of the HPLC peak associated with the same impurity in step (b).

6. A method of determining an amount of an impurity in a sample of donepezil, or a pharmaceutically-acceptable salt thereof as claimed in claim 5 wherein the amount of one or more of following impurities of donepezil is also determined: 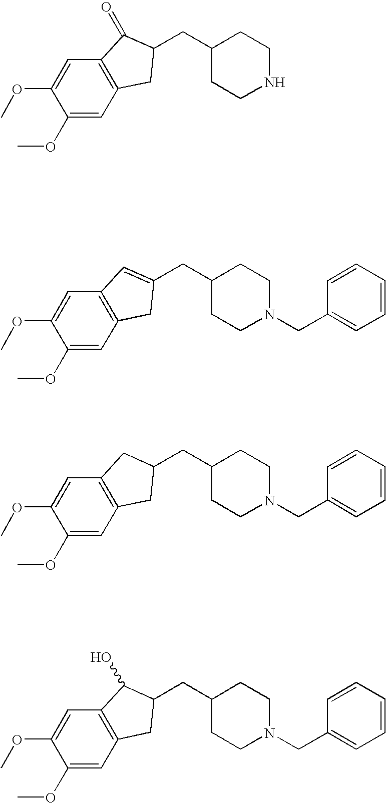
or a diastereomer thereof

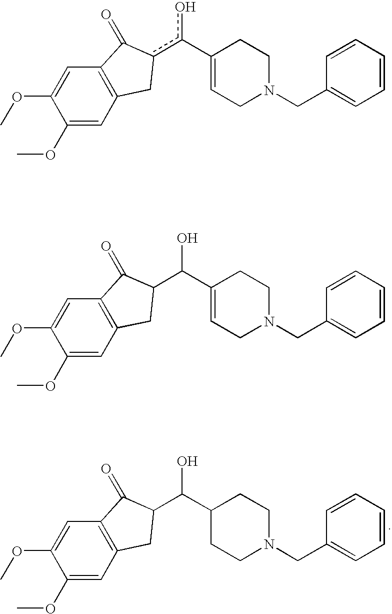

7. A method of determining an amount of an impurity in a sample of donepezil, or a pharmaceutically-acceptable salt thereof, selected from one or more of the following; 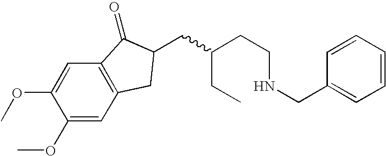
a or a diastereomer thereof, and 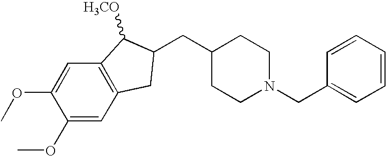
or a diastereomer thereof
the method comprising performing steps (a) and (b) in either order:
(a) subjecting a sample of donepezil, or a pharmaceutically-acceptable salt thereof, comprising an unknown concentration of at least one impurity of donepezil and a sample of a known concentration of at least one impurity of donepezil, as described above, to a chromatographic analysis;
(b) measuring an area of the at least one impurity of donepezil peak obtained from the sample of donepezil, or a pharmaceutically-acceptable salt thereof, and from the sample of the at least one impurity of donepezil;
and calculating a concentration of the at least one impurity of donepezil in the sample of donepezil, or a pharmaceutically-acceptable salt thereof, from the measured area.

8. The method according to claim 7 wherein the chromatographic method is HPLC or TLC analysis.

9. The method according to in claim 8, wherein in both steps (a) and (b) HPLC is carried out.

10. A method of determining an amount of an impurity in a sample of donepezil, or a pharmaceutically-acceptable salt thereof as according to claim 7 wherein the amount of one or more of following impurities of donepezil is also determined: 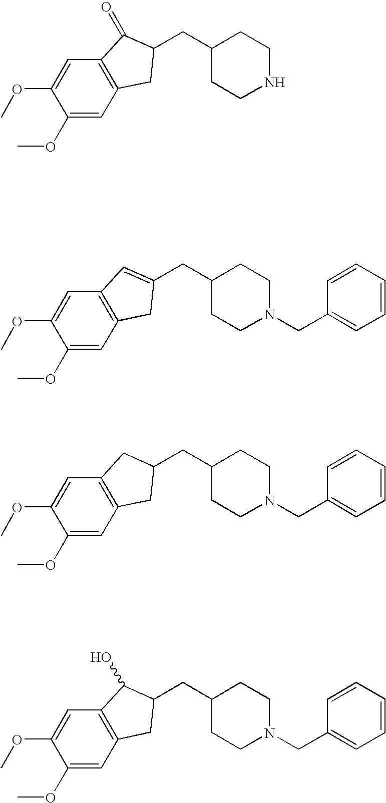
or a diastereomer thereof

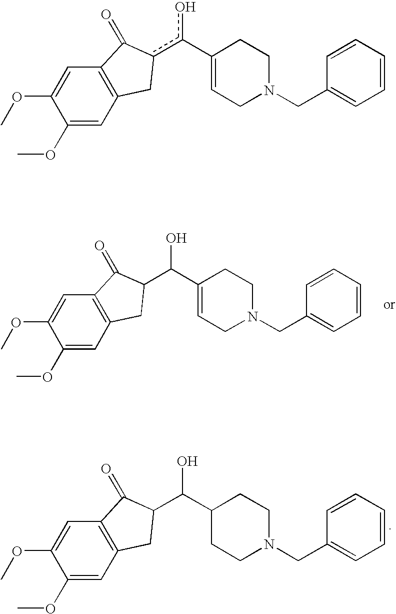

11. A method of determining an amount of an impurity in a sample of donepezil, or a pharmaceutically-acceptable salt thereof, selected from one or more of the following: 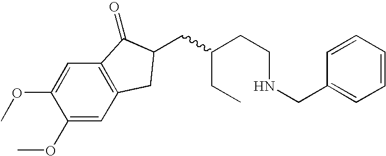
or a diastereomer thereof, and 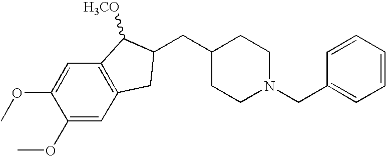
or a diastereomer thereof
the method comprising:
(a) subjecting an aliquot of a reference solution of a known concentration of substantially pure donepezil, or a pharmaceutically-acceptable salt thereof, and an aliquot of reference solution of a known concentration of one substantially pure isolated impurity of donepezil to chromatographic analysis and determining one or more response factors of donepezil, or a pharmaceutically-acceptable salt thereof, and the impurity of donepezil;
(b) subjecting a solution of known overall concentration of donepezil, or a pharmaceutically-acceptable salt thereof, to chromatographic analysis under substantially the same conditions used in step (a);
and calculating an amount of impurity of donepezil in the solution using the respective peak areas and the response factors.

12. The method according to claim 11 wherein the chromatographic analysis is HPLC or TLC analysis.

13. The method according to claim 12, wherein in both steps (a) and (b) HPLC is carried out.

14. A method of determining an amount of an impurity in a sample of donepezil, or a pharmaceutically-acceptable salt thereof according to claim 11 wherein the amount of one or more of following impurities of donepezil is also determined: 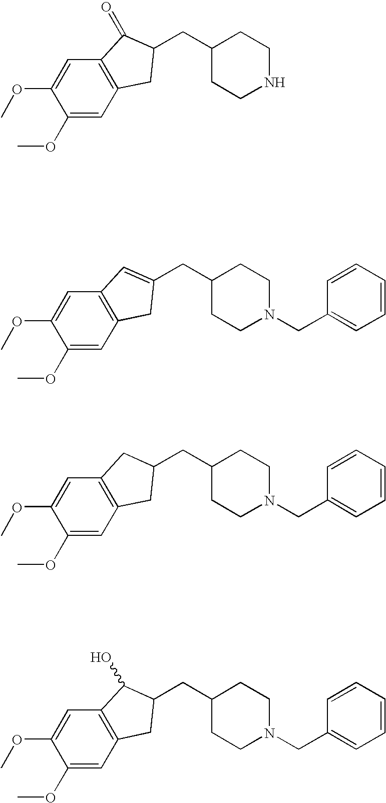
or a diastereomer thereof

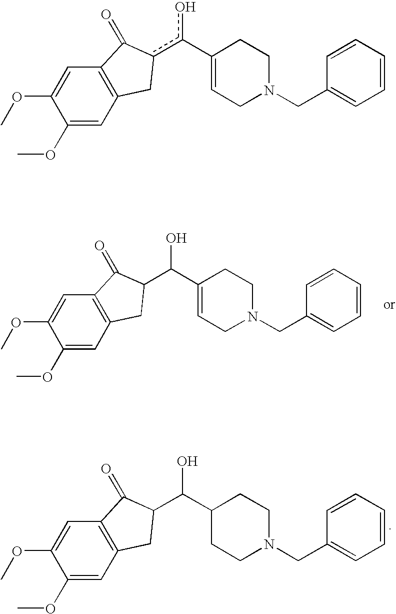

15. The method according to claim 11 wherein the sample of donepezil, or a pharmaceutically-acceptable salt thereof is bulk donepezil, or a pharmaceutically-acceptable salt thereof.

16. The method according to any one of claims 5, 7 or 11 wherein the sample of donepezil, or a pharmaceutically-acceptable salt thereof, is taken from a batch or production lot of donepezil, or a pharmaceutically-acceptable salt thereof, the amount of each impurity of the sample of donepezil is determined to be less than 0.15 area-%.

17. The method according to claim 16, wherein the donepezil is donepezil separated from one or more pharmaceutical dosage forms comprising donepezil.

18. The method according to any one of claims 5, 7 or 11 wherein the donepezil is donepezil separated from one or more pharmaceutical dosage forms comprising donepezil, or a pharmaceutically-acceptable salt thereof.

19. The method according to claim 18 wherein the pharmaceutical dosage forms comprising donepezil, or a pharmaceutically-acceptable salt thereof, is taken from a batch or production lot, the amount of impurity of donepezil is determined to be less than 10 area-%, and the batch is released for sale.

20. A process for preparing a pharmaceutical composition comprising donepezil, or a pharmaceutically-acceptable salt thereof, which comprises formulating donepezil or a pharmaceutically acceptable salt thereof, tested according to any one of the methods according to claims 5, 7 or 11 with an excipient or carrier.

21. An isolated impurity of donepezil, or a pharmaceutically-acceptable salt thereof, having retention time, relative to donepezil, of 1.12±0.01 measured by a method according to claim 1.

22. An isolated impurity of donepezil, or a pharmaceutically-acceptable salt thereof, having retention time, relative to donepezil, of 1.10±10.01 measured by a method according to claim 1.

23. An isolated impurity of donepezil, or a pharmaceutically-acceptable salt thereof having the following structure 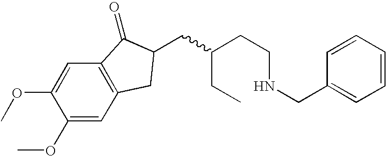
or a diastereomer thereof.

24. An isolated impurity of donepezil, or a pharmaceutically-acceptable salt thereof, having retention time relative to donepezil, of 1.08±0.01 measured by a method of claim 1.

25. An isolated impurity of donepezil, or a pharmaceutically-acceptable salt thereof, having a retention time relative to donepezil, of 1.07±0.02 measured by a method of claim 1.

26. An isolated impurity of donepezil, or a pharmaceutically-acceptable salt having the following structure 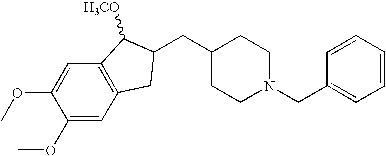
or a diastereomer thereof.

27. One or more isolated impurities of donepezil as according to any one of claims 21 to 26, comprising from 0.03 area-% to 2 area-% of donepezil as judged by HPLC.

28. One or more isolated impurities of donepezil according to any one of claims 21 to 26, comprising 0.05 area-% to 1.5 area-% donepezil as judged by HPLC.

29. An isolated impurity of donepezil, according to any one of claims 21 to 26, which is at least 90 area-% pure as judged by HPLC.

30. Donepezil, or pharmaceutically-acceptable salt thereof, containing one or more impurities according to any one of claims 21 to 26, wherein each impurity is present in an amount not exceeding more than 0.5% wt of the total % wt of donepezil, whether in a form of donepezil or its pharmaceutically-acceptable salt.

31. Donepezil, or pharmaceutically-acceptable salt thereof as claimed in claim 30 further containing one or more of following impurities: 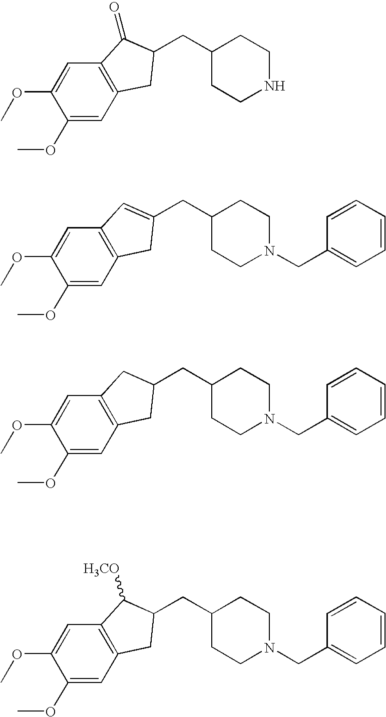
or a diastereomer thereof 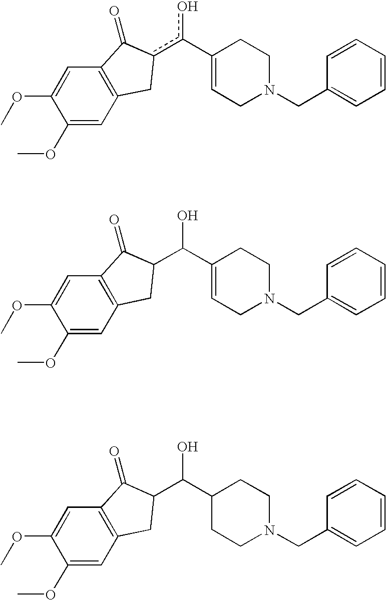
wherein each impurity is present in an amount not exceeding more than 0.5% wt of the total % wt of donepezil, whether in the form of donepezil or its pharmaceutically-acceptable salt.

32. A pharmaceutical composition comprising donepezil, or a pharmaceutically-acceptable salt thereof, and at least one impurity of donepezil according to claim 30.

33. A pharmaceutical composition comprising donepezil, or a pharmaceutically acceptable salt thereof, according to claim 30, wherein the level of each impurity is less than 0.1% wt of the total % wt of donepezil, whether in the form of donepezil or its pharmaceutically-acceptable salt.

34. (canceled)

35. (canceled)
